(19) United States
(12) Patent Application Publication
(10) Pub. No.: US 2021/0401774 A1
(43) Pub. Date: Dec. 30, 2021

(54) KETAMINE PROTOCOLS AND DATA EVALUATION FOR TREATMENT-RESISTANT DEPRESSION AND TRAUMA

(71) Applicant: Robert Brent Turnipseed, Austin, TX (US)

(72) Inventor: Robert Brent Turnipseed, Austin, TX (US)

(21) Appl. No.: 17/356,923

(22) Filed: Jun. 24, 2021

Related U.S. Application Data
(60) Provisional application No. 63/063,847, filed on Aug. 10, 2020.
(60) Provisional application No. 63/045,045, filed on Jun. 27, 2020.

(51) Int. Cl. A61K 31/135; A61P 25/22; A61P 25/24

(52) U.S. Cl. CPC A61K 31/135; A61P 25/24; A61P 25/22

(57) ABSTRACT

In an embodiment, the present disclosure pertains to a method of treatment for treatment-resistant depression or trauma. In general, the method includes administering a dose of ketamine to a subject during at least one session as defined by a ketamine-assisted psychotherapy plan. In some embodiments, the ketamine-assisted psychotherapy plan includes one or more weeks having one or more sessions. In some embodiments, the one or more weeks correspond to a phase of the ketamine-assisted psychotherapy plan. In some embodiments, the dosage of ketamine may be varied between each session of the one or more sessions. In some embodiments, the method further includes conducting at least one baseline assessment before, during, or after at least one of the one or more sessions. In some embodiments, the dose of ketamine is in a range of 0.5 mg/kg to 2.0 mg/kg.

### CROSS-REFERENCE TO RELATED APPLICATIONS

This patent application claims priority from, and incorporates by reference the entire disclosures of, U.S. Provisional Patent Application 63/045,045 filed on Jun. 27, 2020 and U.S. Provisional Patent Application 63/063,847 filed on Aug. 10, 2020.

### TECHNICAL FIELD

The present disclosure relates generally to treatment-resistant depression and more particularly, but not by way of limitation, to a ketamine protocol for treatment-resistant depression and trauma.

### BACKGROUND

This section provides background information to facilitate a better understanding of the various aspects of the disclosure. It should be understood that the statements in this section of this document are to be read in this light, and not as admissions of prior art.

Ketamine-assisted psychotherapy is an innovative approach to psychological change. It combines the benefits of a pharmaceutically-induced neurological and phenomenological shift with the guidance of trained therapists. It can be helpful to think of ketamine as a catalyst for change, with the work of the therapist and client nourishing the specific type of change necessary to reduce suffering and increase psychological freedom.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method of treatment for treatment-resistant depression or trauma. In general, the method includes administering a dose of ketamine to a subject during at least one session as defined by a ketamine-assisted psychotherapy plan. In some embodiments, the ketamine-assisted psychotherapy plan includes one or more weeks having one or more sessions. In some embodiments, the one or more weeks correspond to a phase of the ketamine-assisted psychotherapy plan. In some embodiments, the dosage of ketamine may be varied between each session of the one or more sessions. In some embodiments, the method further includes conducting at least one baseline assessment before, during, or after at least one of the one or more sessions. In some embodiments, the dose of ketamine is in a range of 0.5 mg/kg to 2.0 mg/kg.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Taking an antidepressant or going to psychological counseling (psychotherapy) eases depression and/or trauma symptoms for most people. However, with treatment-resistant depression and trauma, often times standard treatments are not enough. Standard treatments, such as, for example, antidepressant medications and therapy, may not help much at all, or symptoms may improve, only to keep coming back. Psychotherapy by a psychiatrist, psychologist, or other mental health professional can be very effective, and for many people, psychotherapy combined with medications during psychotherapy has been shown to work better than traditional psychotherapy without medication.

Ketamine-assisted psychotherapy, as disclosed herein, is an innovative approach to psychological change that combines the benefits of a pharmaceutically-induced neurological and phenomenological shift with the guidance of trained therapists. This combination generally accelerates the process of psychological growth and change. Ketamine is labelled as a dissociative anesthetic due to its tendency to produce a disconnection from the usual way of feeling and experiencing. At various doses given during a therapy session, this influence can produce shifts in consciousness, such as, for example, expanded states of awareness, novel ways of viewing life concerns, and freedom from the worries and anxieties of ordinary mind-states. Psychotherapy offers real-time reflection on this experience to enhance psychological growth and change. As such, described herein are various ketamine-assisted protocols for the treatment of treatment-resistant depression and trauma.

### Ketamine Protocol for Treatment-Resistant Depression

Baseline Rating Scales for Monitoring Clinical Progress and Response to Treatment. In some embodiments, baseline rating scales can be utilized with ketamine treatment therapies to monitor clinical response and response to treatment. In some embodiments, the baseline rating scales for monitoring clinical progression and response to treatment of depression can include, without limitation, standard depression rating scales (e.g., Hamilton Depression Rating Scale (HAM-D), Montgomery—ÅAsberg Depression Rating Scale (MADRS), and/or Patient Health Questionnaire 9 (PHQ-9)), suicide scales (e.g., Columbia Suicide Severity Rating Scale (C-SSRS) and/or Scale for Suicidal Ideation (SSI)), standard anxiety scales (e.g., General Anxiety Disorder 7 (GAD-7) and/or Beck Anxiety Inventory (BAI)), various other scales if, for example, a patient has another mood comorbidity (e.g., Yale-Brown Obsessive Compulsive Scale (YBOCS) for obsessive-compulsive disorder (OCD)), the Holmes-Raha Life Stess Inventory, and combinations thereof.

Baseline Labs and Predictive Measures. In some embodiments, baseline labs can be used in conjunction with ketamine treatment therapies. In some embodiments, the baseline labs can include screening for contributors of depression. In some embodiments, the screening for contributors of depression can include, without limitation, a complete blood count and/or metabolic panel, thyroid stimulating hormone (TSH) with reflex to T4 testing, B12 testing, folate testing, homocysteine testing, vitamin D testing, methylenetetrahydrofolate reductase (MTHFR) gene level testing, and combinations thereof.

In some embodiments, the baseline labs can include screening for potential predictors of ketamine response. In some embodiments, the screening for predictors of ketamine response can include, without limitation, enzyme-linked immunoassay (ELISA) testing for brain-derived neurotrophic factor (BDNF) val/val homozygotes (possibly better responders) and/or val66met, rs6265, single nucleotide polymorphisms (SNP) testing, c-reactive protein (CRP) testing, interleukin 6 (IL-6) testing, tumor necrosis factor alpha (TNF-a) testing, and combinations thereof.

In some embodiments, low baseline plasma adiponectin levels can predict a rapid response to ketamine. Adiponectin is a cytokine that is secreted by adipocytes and it reduces insulin resistance and exerts anti-inflammatory effects. In some embodiments, after one ketamine treatment, serum kynurenic acid levels greater than 0.532 may show short-term efficacy, as shown in major depressive disorder (MDD) with suicide ideation (SI), for future treatments. Kynurenic acid is a downstream metabolite of tryptophan catabolism, and antagonizes N-methyl-D-aspartic acid (NMDA) and α-amino-3-hydroxy-5-methyl-4-isoxazolepropionic acid (AMPA) receptors.

In some embodiments, a kynurenic to quinolinic acid ratio can be used as a predictor of ketamine response in patients. In some embodiments, baseline D-serine levels are lower in ketamine responders. D-serine is a non-essential amino acid and an agonist at the glycine site of the NMDA receptor.

In some embodiments, therapists can identify if a patient is currently seeking outside therapy. In some embodiments, the therapist can identify co-morbid diagnoses of the patient. In some embodiments, the therapist can identify medical trials the patient has participated in.

Baseline Factors that may be Associated with Ketamine Response. In some embodiments, various baseline factors can be associated with ketamine response and can be utilized in conjunction with ketamine treatment. In some embodiments, the various baseline factors can include, without limitation, baseline body mass index (BMI) and/or metabolic syndrome. In some embodiments, whether or not family history of alcohol use disorder can be a baseline factor. For example, family history of alcohol use disorder can be a baseline factor as evidence that this factor alone may extend antidepressant effect for at least four weeks. Additionally, genetic underpinning may be polymorphism in NR2A subunit of the N-methyl-D-aspartate (NMDA) receptor (typically associated with family history of alcoholism). In some embodiments, assessing baseline insomnia may predict response and remission, as this was calculated using Hamilton Depression Rating Scale 17 (HAMD-17) sleep disruption factor scoring. In some embodiments, assessing baseline neurocognitive performance with a scale that can be tracked can be done via an application, such as, for example, IQ and MATRICS Consensus Cognitive Battery, or other mechanism to measure processing speed.

Screen Out Contradictions. In some embodiments, various screen out contraindications can be identified. For example, in some embodiments, the screen out contradictions can include, without limitation, interstitial cystitis or current urinary tract infection (UTI), pregnancy tests, uncontrolled arrhythmias or hypertension, current mania and/or psychosis, a history of schizophrenia, uncontrolled seizure disorder, substance use considerations, and combinations thereof.

Other Factors that may Optimize Treatment. In some embodiments, other factors that may optimize treatment can include, without limitation, magnesium supplementation, N-acetylcysteine supplementation, optimizing set and setting, taking steps to mitigate anxiety during sessions, combining ketamine with mindfulness-based cognitive therapy, educating patients on mitigating factors (e.g., alcohol, cannabis, benzodiazepines, and/or anticonvulsants usage). In some embodiment, at least some of the aforementioned are used to optimize ketamine treatments.

Treatment Plan. In some embodiments, a treatment plan can include various evaluations appointment. In some embodiments, an informed consent is conducted during the patient's second appointment. In some embodiments, various therapist preparations can occur during the patient's third appointment. In some embodiments, for example if trauma is present, therapist preparation can occur more than once, for example, in multiple sessions. In some embodiments, once dissociation has been achieved as measured by the Clinician-Administered Dissociative States Scale (CADSS), consideration for dosing for subsequent sessions can be considered and/or evaluated. In some embodiments, treatment-emergent manic symptoms can be assessed using, for example, but without limitation, the Young Mania Rating Scale.

Data Collection/Potential Candidate Evaluation. In various embodiments the present disclosure pertains to detailed protocols aimed to optimize ketamine treatment using a graduated dosing schedule. During the sessions, as outlined in detail below, various measures can be used to gauge the response of the ketamine treatment. As such, another aspect of the present disclosure pertains to the collection of biomarkers and historical factors, such as, but not limited to, a family history of alcohol use disorder, that when used in combination with the ketamine treatment plans can be an invaluable tool used to help clinicians or entities (e.g., clinics, insurance companies, doctors, and the like) determine patients and/or patient types that are more likely to benefit, or respond, to ketamine treatments. Accordingly, information gathered can be utilized to identify potential candidates for the ketamine treatment plans as disclosed herein.

In this manner, in some embodiments, the treatment plans as disclosed herein can further include collecting treatment-related data from the subject before, during, or after the sessions as outline in detail below. In some embodiments, the treatment-related data includes information obtained during a baseline assessment. In some embodiments, the treatment-related data includes progress of the subject based, at least in part, on an assessment of the subject before, during, or after each session of the treatment plans presented herein. In some embodiments, the methods of the present disclosure can further include identifying potential candidates for the ketamine-assisted psychotherapy plan based, at least in part, on the treatment-related data. In this fashion, the treatment-related data can be used to identify potential candidates for clinics, insurance companies, doctors, and the like.

### Example Treatment Plan (Treatment-Resistant Depression)

Reference will now be made to more specific embodiments of the present disclosure. However, it should be noted that the disclosure below is for illustrative purposes only and is not intended to limit the scope of the claimed subject matter in any way.

Presented below is a multi-week treatment plan for ketamine application therapy. In some embodiments, doses listed can be the same for both intravenous (IV) routes and intramuscular (IM) routes of administration. Additional administration types are readily envisioned.

### Weeks 1-4: Induction Phase

Presented below is an example induction phase corresponding to four weeks. In some embodiments, the induction phase can be conducted in fewer or more weeks as deemed by a therapist and patient response.

Week 1, Session 1. 0.5 mg/kg ketamine dosage is utilized and CADSS evaluation is conducted. In some embodiments, the therapist can consider focusing only on depersonalization subscales. In some instances, depersonalization subscales can also utilize simple direct questioning about the presence of dissociation (e.g., yes or no to dissociation verses degree of dissociation). In some instances, at 24 hours, if the patient remits, a therapist can consider changing course to once per week for two weeks, then maintenance every two to four weeks. Additionally, in some embodiments, the therapist can look for improvements in specific depression symptoms (e.g., reported sadness, apparent sadness, inability to feel, difficulty concentrating, and combinations of the same and like) that may indicate the ability to have treatments no more frequent than every two weeks. In some embodiments, the therapist records qualitative data and changes in life events after each session. In some embodiments, the ketamine dosage in Session 1 can be in a range of 0.5 mg/kg to 2 mg/kg.

Week 1, Session 2. 0.75 mg/kg ketamine dosage can be utilized and CADSS evaluation can be conducted. In some embodiments, during the patient appointment, the therapist can consider tracking cognitive performance. In some embodiments, before, during, and/or after Session 1 or 2, the therapist can consider various depression scales, anxiety scales, suicide scales, and combinations thereof. In some embodiments, the ketamine dosage in Session 2 can be in a range of 0.5 mg/kg to 2 mg/kg.

Week 2, Session 3. 1 mg/kg ketamine dosage can be utilized and CADSS evaluation can be conducted. In some embodiments, the ketamine dosage in Session 3 can be in a range of 0.5 mg/kg to 2 mg/kg.

Week 2, Session 4. 1.25 mg/kg ketamine dosage can be utilized and CADSS evaluation can be conducted. In some embodiments, before, during, and/or after Sessions 3 and/or 4, the therapist can track cognitive performance, depression, anxiety, suicide scale, and combinations thereof. In some embodiments, the ketamine dosage in Session 4 can be in a range of 0.5 mg/kg to 2 mg/kg.

Week 3, Session 5. 1.5 mg/kg ketamine dosage can be utilized and CADSS evaluation can be conducted. In some embodiments, the ketamine dosage in Session 5 can be in a range of 0.5 mg/kg to 2 mg/kg.

Week 3, Session 6. 1.75 mg/kg ketamine dosage can be utilized and CADSS evaluation can be conducted. In some embodiments, before, during, and/or after Sessions 5 and/or 6, the therapist can track cognitive performance, depression, anxiety, suicide scale, and combinations thereof. In some embodiments, the ketamine dosage in Session 6 can be in a range of 0.5 mg/kg to 2 mg/kg.

Week 4, Session 7. 2 mg/kg ketamine dosage can be utilized and CADSS evaluation can be conducted. In some embodiments, the ketamine dosage in Session XX can be in a range of 0.5 mg/kg to 7 mg/kg.

Week 4, Session 8. 2 mg/kg ketamine dosage can be utilized and CADSS evaluation can be conducted. In some embodiments, before, during, and/or after Sessions 7 and/or 8, the therapist can track cognitive performance, depression, anxiety, suicide scale, and combinations thereof. Additionally, in some embodiments, before, during, and/or after Sessions 7 and/or 8, the therapist can repeat any or all baseline lab work for the patient. For example, in some embodiments, the repeated baseline lab work can include, without limitation, any labs that had previously been out of range or suboptimal. In some embodiments, the therapist can repeat TSH testing, folate testing, B12 testing, homocysteine testing, CRP testing, IL-6 testing, TNF-a testing, vitamin D testing, and combinations thereof. In some embodiments, the ketamine dosage in Session 8 can be in a range of 0.5 mg/kg to 2 mg/kg.

### Weeks 5-8: Step-Down Phase

Presented below is an example step-down phase corresponding to four weeks. In some embodiments, the step-down phase can be conducted in fewer or more weeks as deemed by a therapist and patient response.

Week 5. In some embodiments, dosage can remain constant during Week 5. In some embodiments, dosage can vary during Week 5 (e.g., no dose, higher dose, or lower dose). In some embodiments, during Week 5, in-office psychiatric follow-up appointments can be conducted. In some embodiments, during Week 5, one-hour therapy session with integration and outcome assessments can be conducted. In some embodiments, during Week 5, a one hour integration session can be conducted with the therapist. In some embodiments, during Week 5, the therapist can track cognitive performance, depression, anxiety, suicide scale, and combinations thereof.

Week 6, Session 9. 2 mg/kg ketamine dosage can be utilized and CADSS evaluation can be conducted. In some embodiments, before, during, and/or after Session 9, the therapist can track cognitive performance, depression, anxiety, suicide scale, and combinations thereof. In some embodiments, the ketamine dosage in Session 9 can be in a range of 0.5 mg/kg to 2 mg/kg.

Week 7. In some embodiments, dosage can remain constant during Week 7. In some embodiments, dosage can vary during Week 7 (e.g., no dose, higher dose, or lower dose). In some embodiments, during Week 7, in-office psychiatric follow-up appointments can be conducted. In some embodiments, during Week 7, one-hour therapy session with integration and outcome assessments can be conducted. In some embodiments, during Week 7, the therapist can track cognitive performance, depression, anxiety, suicide scale, and combinations thereof.

Week 8, Session 10. 2 mg/kg ketamine dosage can be utilized and CADSS evaluation can be conducted. In some embodiments, the ketamine dosage in Session 10 can be in a range of 0.5 mg/kg to 2 mg/kg.

### Week 9 and Beyond: Maintenance Phase

Presented below is an example maintenance phase corresponding to time after Week 8. In some embodiments, the maintenance phase can be conducted in fewer or more weeks as deemed by a therapist and patient response.

Week 9. In some embodiments, during Week 9 (or beyond), an in-office follow up medical appointment is conducted. In some embodiments, Ketamine-Assisted Psychotherapy (KAP) begins maintenance. In some embodiments, KAP maintenance is determined during any of the past weeks based on therapist assessment and patient response. In some embodiments, the maintenance phase is determined in Weeks 2, 3, and/or 4 (or any preceding week). It should be noted that average time to relapse is approximately 19 to 22 days. In some embodiments, during Week 9 (or beyond), the therapist can repeat any or all baseline lab work for the patient. For example, in some embodiments, the repeated baseline lab work can include, without limitation, any labs that had previously been out of range or suboptimal. In some embodiments, the therapist can repeat TSH testing, folate testing, B12 testing, homocysteine testing, CRP testing, IL-6 testing, TNF-a testing, vitamin D testing, and combinations thereof. In some embodiments, the ketamine dosage in any session can be in a range of 0.5 mg/kg to 2 mg/kg.

Variations to Treatment Schedule. In some embodiments, the treatment schedule identified above can be modified or altered according to therapist assessment and patient response. For example, in some embodiments, one variation can include two treatments per week for three weeks, then once per week for two weeks, then once every other week two times (2×), then maintenance can be conducted every two to four weeks. In some embodiments, if remission is achieved at any point in the treatment, then the treatment plan can advance to one treatment per week for two weeks, then one treatment every two weeks two times (2×), then maintenance is every two to four weeks.

### Ketamine Protocol for Trauma

Disclosed herein is a treatment protocol guided by existing evidence for use of ketamine for various trauma-related conditions. Generally, studies have focused on the standard use of 0.5 mg/kg over 40 minutes (IV) in treating mood disorders. However, there is evidence of safe treatment and numerous benefits of doses above the standard (e.g., dosing above 0.5 mg/kg). The protocol herein is intended to be used to treat post-traumatic stress disorder (PTSD) and other trauma-related symptoms and/or clinical presentations.

Vital Signs. In some embodiments, various vital signs can be monitored in conjunction with ketamine treatments before, during, and/or after treatment. For example, in some embodiments the vital signs can include, without limitation, weight in kilograms and height to calculate BMI, blood pressure, heart rate, pulse oximetry, and combinations of the same and like. In some embodiments, these vital signs can be taken once, prior to initiating ketamine treatment (e.g., during intake or informed consent appointments). In some embodiments, at each ketamine treatment session, blood pressure, heart rate, and pulse oximetry can be checked pre-treatment, and then re-checked once more prior to discharge from the clinic.

In some embodiments, prior to ketamine treatment, if systolic blood pressure is greater than 180 or diastolic blood pressure is greater than 110, the patient can be referred to a primary care physician for treatment of hypertension. In some embodiments, during a ketamine treatment session, if systolic blood pressure is greater than 180 or diastolic blood pressure is greater than 110, a patient may be treated with clonidine (e.g., 0.1 mg per os (PO)×1). In some instances, the patient can then wait 20-30 minutes and a re-check of blood pressure can be performed. In some instances, it is ideal to not administer ketamine at this time, but to instead refer the patient to a primary care physician to manage blood pressure. In cases of IV ketamine treatment, reduction of blood pressure can be achieved through use of labetalol IV 20 mg over 1 minute (ideally, not faster), or for chronic obstructive pulmonary disease (COPD) and/or asthma, metoprolol 5 mg over 1 minute (ideally, no faster) may be used. In some embodiments, IV diltiazem 0.25 mg/kg over 1 minute may be used if the patient is unable to tolerate beta blockers. In some embodiments, administration of labetalol or diltiazem can be repeated every 10 minutes as needed, and metoprolol may be repeated every 5 minutes as needed. In general, systolic blood pressure should be less than 180 prior to discharge, and diastolic blood pressure should be less than 110 prior to discharge.

In some embodiments, if heartrate is greater than 110 bpm during ketamine treatment, the patient can be treated with clonidine (e.g., 0.1 mg×1), and vitals re-checked after waiting 20-30 minutes. In embodiments utilizing IV treatment, labetalol or metoprolol may be used as described above with respect to blood pressure. Generally, heartrate should be less than 110 bpm prior to discharge. In general, pulse oximetry readings should be at least 92% in order to administer ketamine, and prior to patient discharge.

Infusion Times. In some embodiments, for IV ketamine treatment, doses of 50 mg and less can generally be infused for 40 minutes. In some embodiments, for IV ketamine treatment, doses of 50-75 mg can generally be infused for 45 minutes. In some embodiments, for IV ketamine treatment, doses of 75-100 mg can generally be infused for 50 minutes. In some embodiments, for IV ketamine treatment, doses exceeding 100 mg can generally be infused for 55 minutes. However, in some embodiments, the total infusion time can be adjusted to fit the needs and treatment response best suited to the patient. In some embodiments, infusion times are not less than 40 minutes.

In general, if psychiatric emergencies arise during the course of the treatment (e.g., emergent SI, worsening of SI, mania, and the like), rapid follow-up evaluation by a physician assistant/associate or medical doctor might be necessary (generally, less than one week) and/or referral to inpatient treatment, if appropriate.

Nausea. In some embodiments, pre-treatment for potential nausea may be administered using, for example, 8-16 mg sublingual administration (SL) ondansetron, and repeated as necessary. In some embodiments, IV ondansetron 4 mg can be administered over 10 seconds prior to beginning infusion, and can be repeated at a dose of 4 mg, as needed, for emergent nausea.

Anxiety and/or Agitation. In some embodiments, for IM ketamine treatment, an orally disintegrating tablet (ODT) of clonazepam (e.g., 0.25-0.5 mg) can be administered every 30 minutes as needed to counter anxiety or agitation. In some embodiments, lorazepam 0.5-1 mg every 30 minutes can be administered as needed. In some embodiments, for IV ketamine treatment, lorazepam 2 mg over 1 minute can be administered and may, in some instances, be repeated in 10 minute increments, as needed.

Psychotherapy Modality. While psychotherapy is often tailored to the experience and treatment goals of each individual, the protocols disclosed herein depend, at least in part, on the therapist utilizing modalities that are especially “trauma informed’, notably eye movement desensitization and reprocessing (EMDR), cognitive processing, psychodynamic psychotherapy, and combinations of the same and like.

Baseline Rating Scales for Monitoring Clinical Progress and Response to Treatment. In some embodiments, baseline rating scales for monitoring clinical progress and response to treatment can utilized during ketamine treatment. In some embodiments, the baseline rating scales can include, for example, trauma scales, such as, the PTSD checklist civilian version (PCL-C), or the clinician-administered PTSD scale for Diagnostic and Statistical Manual of Mental Disorders, 5th Edition (CAPS-5), and combinations of the same and like. In some embodiments, baseline rating scales for monitoring clinical progress and response to treatment can include, for example, standard depression rating scales, such as, HAM-D, MADRS, the quick inventory of depressive symptomatology (QIDS), PHQ-9, and combinations of the same and like. In some embodiments, baseline rating scales for monitoring clinical progress and response to treatment can include, for example, standard anxiety scales, such as, GAD-7, BAI, and combinations of the same and like.

Baseline Labs—Screening for Potential Contributors of Mood Symptoms. In some embodiments, the baseline labs can be utilized to screen for potential contributors of mood symptoms. For example, in some embodiments, the baseline labs can include, without limitation, a complete blood count, a complete metabolic panel, thyroid stimulating hormone with reflex to T4 testing, B12 testing, folate testing, homocysteine testing, methylmalonic acid testing, vitamin D testing, MTHFR gene level testing, CRP testing (e.g., to screen for chronic inflammation), IL-6 testing, TNF-a testing, and combinations of the same and like.

Baseline Factors Associated with Ketamine Response. In some embodiments, patients can be tested to predict the likelihood of benefits realized through ketamine treatment. In some embodiments, the baseline factors can include, for example, a baseline BMI or identification as to whether or not the patient has a family history of alcohol use disorder, as evidence shows that this factor alone may extend antidepressant effect for at least four weeks. In some embodiments, genetic underpinning may be polymorphism in NR2A subunit of the NMDA receptor, which can be associated with a family history of alcoholism which can indicate potential ketamine response. In some embodiments, assessments of baseline neurocognitive performance (e.g., MATRICS battery) can be conducted, where, in general, lower scores are typically associated with a better response to ketamine treatment.

In some embodiments, ELISA testing for BDNF val/val homozygotes (better responders) and val66met rs6265 SNP (less likely to respond) can be conducted to identify potential response in the patient. Furthermore, cytokines can be utilized as a bassline factor utilizing CRP testing. Addition testing can include, but is not limited to, IL-6 and TNF-a testing to determine baseline factors. In some embodiments, baseline factors can additionally include, for example, plasma adiponectin levels, serum kynurenic acid levels (generally following the first ketamine treatment), kynurenic to quinolinic acid ratios, D-serine levels, and combinations of the same and like.

Screen Out Contraindications. In some embodiments, various screen out conditions can be identified. For example, in some embodiments, screen out conditions can include, without limitation, interstitial cystitis or a current UTI, pregnancy (by history or via a urinary pregnancy test) or nursing, unstable cardiovascular diseases (e.g., uncontrolled arrhythmias or hypertension, recent myocardial infarction, recent transient ischemic attack, or stroke), current mania and/or psychosis, a history of schizophrenia, uncontrolled seizure disorder, severe and/or current substance use, addiction or substance use disorder (e.g., intoxication or withdrawal syndrome less than one week prior to treatment), and combinations thereof.

Other Factors that may Optimize Treatment. In some embodiments, other factors that may optimize ketamine treatment can include, without limitation, magnesium supplementation, N-acetylcysteine supplementation, attention to set and setting, taking steps to reduce anxiety during sessions, patient education regarding factors that may reduce efficacy of ketamine (e.g., alcohol, cannabis, benzodiazepines or anticonvulsants usage, living in an unstable or dangerous environment, and suboptimal treatment schedules). In some embodiments, the aforementioned factors can be addressed before, during, and/or after ketamine treatment.

Treatment Plan and Dosing and Scale Notes. In general, two treatments per week during the induction phase is optimal. It should be noted that if patients are unable to complete two sessions per week, they should be counseled that they may risk having a suboptimal, or delayed, response. In some embodiments, treatment-emergent manic symptoms may be assessed using the Young Mania Rating Scale.

Dosing Note 1—Response Indicator. Dissociation has been correlated with treatment response in MDD. However, it is unclear yet if this applies to PTSD treatment as well. However, changes in primary mood scales are perhaps the most reliable benchmark regarding treatment response. If the patient, medical provider, and/or therapist agree that the patient has adequately responded to the treatment, the dose does not need to be increased (if there is still room to increase per the protocol schedule).

Dosing Note 2—Inherent Differences in Routes. Dosing per protocol can be the same for both IV and IM routes. However, given that peak plasma levels will be higher for IV versus IM, clinical response might occur at a lower dose with IV.

Dosing Note 3—Dosing Limitation. While the dosing protocols presented herein are likely to be effective for most patients, there can be some who respond to doses below or above the recommended schedules. As such, each provider may adjust the dose to a small degree depending on the clinical scenario. However, a provider should not deviate greater than 0.15 mg/kg change from the scheduled doses without appropriate approval and consideration.

Dosing Note 4—Switching Routes. If switching from IM to IV, calculate the weight-based ratio in mg/kg, and then reduce the dose by 0.15 mg/kg. If switching from IV to IM, calculate the weight-based ratio in mg/kg, and then increase the dose by 0.15 mg/kg.

Dosing Note 5—Split Dosing. This approach may be used for IM sessions in which the patient has difficulty tolerating the dissociative effects of ketamine, for example, an overly anxious reaction. In this instance, one-half the total dose recommended can be given IM, followed by the other one-half ten minutes later.

If a patient has not responded as evidenced by a primary trauma scale (at least 30% reduction in severity from baseline) by Session 6, a medical provider can discuss options with the patient, as ketamine may not ultimately be an effective treatment.

Additionally, patients who have previously had IM or IV ketamine with another facility or provider, dosing can depend on the circumstances of each individual. For example, if the patient had been in maintenance and doing well, continuation of their most recent effective dose may be prescribed. In another example, if the patient had relapsed, in PTSD for example, and their current episode has been present for two months, re-starting an induction with initial dosing recommendations may be considered—though reducing the induction total sessions from six to four may also be considered. It should be noted that these situations are highly variable, and each provider has discretion to tailor the treatment to the needs of the patient. Additionally, ketamine treatment records from other providers can be requested and referenced or considered as needed.

Scales Note. Patients can be given mood scales to complete when they check in for each ketamine treatment session. In some embodiments, the mood scales can include, without limitation, PCL-C and PHQ-9 scales. However, other scales may be used depending on various conditions to be monitored.

Additionally, it should be noted that some patients may request ketamine treatment who are already taking intranasal, oral, or sublingual ketamine at home from a referring provider. In general, it is best to recommend that the patient cease their at-home ketamine at least 48 hours prior to beginning ketamine treatments. Patients should also refrain from taking any at-home ketamine during the treatment schedules provided herein. This is to reduce the chance of tolerance which may comprise the benefit scheduled treatments. However, depending on the circumstances, some patients may be allowed to resume their at-home ketamine as part of the maintenance plan, though this may or may not be necessary.

### Example Treatment Plan (Trauma)

Reference will now be made to more specific embodiments of the present disclosure. However, it should be noted that the disclosure below is for illustrative purposes only and is not intended to limit the scope of the claimed subject matter in any way.

Presented below is a multi-week treatment plan for ketamine application therapy. In some embodiments, doses listed can be the same for both intravenous (IV) routes and intramuscular (IM) routes of administration. Additional administration types are readily envisioned.

### Pre-Induction Phase

Presented below is an example pre-induction phase corresponding to two appointments. In some embodiments, the pre-induction phase can be conducted in fewer or more appointments as deemed by a therapist.

Appointment 1. Initial evaluation with a medical provider (medical doctor or physician assistant/associate).

Appointment 2. Therapist preparation. In some embodiments, potentially more sessions can be required depending on psychiatric history.

### Weeks 1-3: Induction Phase

Presented below is an example induction phase corresponding to three weeks. In some embodiments, the induction phase can be conducted in fewer or more weeks as deemed by a therapist and patient response.

Week 1, Session 1. 0.5 mg/kg ketamine dosage is administered. In some embodiments, a ketamine dissociation scale (KDS) is conducted just prior to the patient leaving. In some embodiments, the ketamine dosage in Session 1 can be in a range of 0.5 mg/kg to 2 mg/kg.

Week 1, Session 2. 0.75 mg/kg ketamine dosage is administered. In some embodiments, a KDS is subsequently administered. In some embodiments, the ketamine dosage in Session 2 can be in a range of 0.5 mg/kg to 2 mg/kg.

Week 2, Session 3. 0.75 mg/kg ketamine dosage is administered. In some embodiments, a KDS is subsequently administered. In some embodiments, the ketamine dosage in Session 3 can be in a range of 0.5 mg/kg to 2 mg/kg.

Week 2, Session 4. 1.0 mg/kg ketamine dosage is administered. In some embodiments, a KDS is subsequently administered. In some embodiments, the ketamine dosage in Session 4 can be in a range of 0.5 mg/kg to 2 mg/kg.

Week 3, Session 5. 1.0 mg/kg ketamine dosage is administered. In some embodiments, a KDS is subsequently administered. In some embodiments, the ketamine dosage in Session 5 can be in a range of 0.5 mg/kg to 2 mg/kg.

Week 3, Session 6. 1.25 mg/kg ketamine dosage (or highest dose needed for response) is administered. In some embodiments, a KDS is subsequently administered. In some embodiments, the ketamine dosage in Session 6 can be in a range of 0.5 mg/kg to 2 mg/kg.

In some embodiments, if the patient has improved, though not fully responded, the provider can recommend, for example, two additional ketamine treatments as part of the induction phase. In some embodiments, doses will not be increase beyond 1.25 mg/kg without required approval and consideration. However, in some embodiments, the ketamine dosage in any session can be in a range of 0.5 mg/kg to 2 mg/kg.

### Weeks 4-7: Step-Down Phase

Presented below is an example step-down phase corresponding to four weeks. In some embodiments, the step-down phase can be conducted in fewer or more weeks as deemed by a therapist and patient response.

Week 4, Session 7. 1.25 mg/kg ketamine dosage (or highest dose needed for response) is administered. In some embodiments, a KDS is subsequently administered. In some embodiments, a psychiatric follow-up appointment to evaluate progress can be conducted before, during, or after Session 7. In some embodiments, the ketamine dosage in Session 7 can be in a range of 0.5 mg/kg to 2 mg/kg.

Week 5, Session 8. 1.25 mg/kg ketamine dosage (or highest dose needed for response) is administered. In some embodiments, a KDS is subsequently administered. In some embodiments, the ketamine dosage in Session 8 can be in a range of 0.5 mg/kg to 2 mg/kg.

Week 6. In some embodiments, no treatment occurs during Week 6. For instance, in some embodiments, Week 6 can include, for example, a tapering process.

Week 7, Session 9. 1.25 mg/kg ketamine dosage (or highest dose needed for response) is administered. In some embodiments, a KDS is subsequently administered. In some embodiments, an in-office medical follow-up appointment is conducted before, during, or after Session 9. In some embodiments, the ketamine dosage in Session 9 can be in a range of 0.5 mg/kg to 1.25 mg/kg.

In some embodiments, the next ketamine treatment can begin the maintenance phase. In some embodiments, the medical provider can evaluate the treatment summary to this point and discuss with the patient the proposed initial maintenance schedule. In some embodiments, this can be once every 3-4 weeks. In some embodiments, baseline labs can be repeated and evaluated. In some embodiments, baseline labs that had previously been out of range or suboptimal are repeated and evaluated. For example, in some embodiments, TSH testing, folate testing, B12 testing, homocysteine, CRP testing, IL-6 testing, TNF-a, testing vitamin D testing, and combinations thereof are repeated.

Week 8. In some embodiments, no treatment occurs during Week 8. For instance, in some embodiments, Week 8 can include, for example, a tapering process.

Week 9. In some embodiments, no treatment occurs during Week 9. For instance, similar to Week 8, in some embodiments, Week 9 can include, for example, an additional tapering process.

### Week 10 and Beyond: Maintenance Phase

Week 10, Session 10. 1.25 mg/kg ketamine dosage (or highest dose needed for response) is administered. In some embodiments, a KDS is subsequently administered. In some embodiments, maintenance schedules can be one session every 2-4 weeks, depending on clinical progress to this point. In some embodiments, patients may be able to space out maintenance sessions beyond every 4 weeks. In some embodiments, spacing out maintenance schedules can be assessed at psychiatric follow-up appointments. In some embodiments, the ketamine dosage in Session 10 can be in a range of 0.5 mg/kg to 2 mg/kg. In some embodiments, the ketamine dosage in any session can be in a range of 0.5 mg/kg to 2 mg/kg.

In view of the aforementioned, in an aspect, the present disclosure pertains to a method of treatment for treatment-resistant depression and/or trauma. In general, the method includes administering a dose of ketamine to a subject during at least one session as defined by a ketamine-assisted psychotherapy plan. In some embodiments, the ketamine-assisted psychotherapy plan includes one or more weeks having one or more sessions. In some embodiments, the one or more weeks correspond to a phase of the ketamine-assisted psychotherapy plan. In some embodiments, the dosage of ketamine may be varied between each session of the one or more sessions. In some embodiments, the method further includes conducting at least one baseline assessment before, during, or after at least one of the one or more sessions. In some embodiments, the dose of ketamine is in a range of 0.5 mg/kg to 2.0 mg/kg.

In some embodiments, the one or more weeks correspond to: (1) an induction phase covering week 1, week 2, week 3, and week 4; (2) a step-down phase covering week 5, week 6, week 7, and week 8; and (3) a maintenance phase covering at least week 9.

In some embodiments, week 1 includes session 1 and session 2, and the dose of ketamine for session 1 is 0.5 mg/kg and the dose of ketamine for session 2 is 0.75 mg/kg. In some embodiments, week 2 includes session 3 and session 4, and the dose of ketamine for session 3 is 1 mg/kg and the dose of ketamine for session 4 is 1.25 mg/kg. In some embodiments, week 3 includes session 5 and session 6, and the dose of ketamine for session 5 is 1.5 mg/kg and the dose of ketamine for session 6 is 1.75 mg/kg. In some embodiments, week 4 includes session 7 and session 8, and the dose of ketamine for session 7 is 2 mg/kg and the dose of ketamine for session 8 is 2 mg/kg. In some embodiments, the dose of ketamine for week 5 is less than, greater than, or equal to the dose of ketamine in at least one of week 1, week 2, week 3, or week 4. In some embodiments, week 6 includes session 9, and the dose of ketamine for session 9 is 2 mg/kg. In some embodiments, the dose of ketamine for week 7 is less than, greater than, or equal to the dose of ketamine in at least one of week 1, week 2, week 3, week 4, week 5, or week 6. In some embodiments, week 8 includes session 10, and the dose of ketamine for session 10 is 2 mg/kg. In some embodiments, week 9 includes an assessment to determine a maintenance dose of ketamine.

In some embodiments, the one or more weeks correspond to: (1) a pre-induction phase having one or more appointments for a medical evaluation of the subject and therapist preparation; (2) an induction phase covering week 1, week 2, and week 3; (3) a step-down phase covering week 4, week 5, and week 7; and (4) a maintenance phase covering at least week 10.

In some embodiments, week 1 includes session 1 and session 2, and the dose of ketamine for session 1 is 0.5 mg/kg and the dose of ketamine for session 2 is 0.75 mg/kg. In some embodiments, week 2 includes session 3 and session 4, and the dose of ketamine for session 3 is 0.75 mg/kg and the dose of ketamine for session 4 is 1.0 mg/kg. In some embodiments, week 3 includes session 5 and session 6, and the dose of ketamine for session 5 is 1.0 mg/kg and the dose of ketamine for session 6 is at least 1.25 mg/kg. In some embodiments, week 4 includes session 7, and the dose of ketamine for session 7 is at least 1.25 mg/kg. In some embodiments, week 5 includes session 8, and the dose of ketamine for session 8 is at least 1.25 mg/kg. In some embodiments, week 7 includes session 9, and the dose of ketamine for session 9 is at least 1.25 mg/kg. In some embodiments, week 10 includes session 10, and the dose of ketamine for session 10 is at least 1.25 mg/kg.

In some embodiments, the at least one baseline assessment can include, without limitation, a baseline rating scales for monitoring clinical progression and response to treatment of depression, standard depression rating scales, the Hamilton Depression Rating Scale (HAM-D), the Montgomery—Åsberg Depression Rating Scale (MADRS), the Patient Health Questionnaire 9 (PHQ-9), suicide scales, the Columbia Suicide Severity Rating Scale (C-SSRS), the Scale for Suicidal Ideation (SSI), standard anxiety scales, the General Anxiety Disorder 7 (GAD-7), the Beck Anxiety Inventory (BAI), the Yale-Brown Obsessive Compulsive Scale (YBOCS) for obsessive-compulsive disorder (OCD), the Holmes-Raha Life Stess Inventory, cognitive performance evaluations, depression evaluations, anxiety evaluations, suicide scale evaluations, the post-traumatic stress disorder (PTSD) checklist civilian version (PCL-C), the Clinician-Administered PTSD Scale for Diagnostic and Statistical Manual of Mental Disorders 5th Edition (CAPS-5), the quick inventory of depressive symptomatology (QIDS), a complete blood count panel, a metabolic panel, thyroid stimulating hormone (TSH) with reflex to T4 testing, B12 testing, folate testing, homocysteine testing, vitamin D testing, methylenetetrahydrofolate reductase (MTHFR) gene level testing, enzyme-linked immunoassay (ELISA) testing for brain-derived neurotrophic factor (BDNF) val/val homozygotes, val66met or rs6265, single nucleotide polymorphisms (SNP) testing, c-reactive protein (CRP) testing, interleukin 6 (IL-6) testing, tumor necrosis factor alpha (TNF-a) testing, plasma adiponectin level testing, serum kynurenic acid level testing, kynurenic to quinolinic acid ratio testing, D-serine level testing, a ketamine dissociation scale, and combinations thereof.

In some embodiments, the method further includes collecting treatment-related data from the subject before, during, or after at least one of the one or more sessions. In some embodiments, the treatment-related data includes at least one of information obtained during the at least one baseline assessment or progress of the subject based, at least in part, on an assessment of the subject before, during, or after each session of the one or more sessions. In some embodiments, the method further includes, identifying potential candidates for the ketamine-assisted psychotherapy plan based, at least in part, on the treatment-related data.

Although various embodiments of the present disclosure have been described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A method of treatment for treatment-resistant depression or trauma, the method comprising:
administering a dose of ketamine to a subject during at least one session as defined by a ketamine-assisted psychotherapy plan, the ketamine-assisted psychotherapy plan comprising:
one or more weeks comprising one or more sessions;
wherein the one or more weeks correspond to a phase of the ketamine-assisted psychotherapy plan; and
wherein the dosage of ketamine may be varied between each session of the one or more sessions;
conducting at least one baseline assessment before, during, or after at least one of the one or more sessions; and
wherein the dose of ketamine is in a range of 0.5 mg/kg to 2.0 mg/kg.

2. The method of claim 1, wherein the one or more weeks correspond to:
an induction phase comprising week 1, week 2, week 3, and week 4;
a step-down phase comprising week 5, week 6, week 7, and week 8; and
a maintenance phase comprising at least week 9.

3. The method of claim 2, wherein week 1 comprises session 1 and session 2, and wherein the dose of ketamine for session 1 is 0.5 mg/kg and the dose of ketamine for session 2 is 0.75 mg/kg.

4. The method of claim 2, wherein week 2 comprises session 3 and session 4, and wherein the dose of ketamine for session 3 is 1 mg/kg and the dose of ketamine for session 4 is 1.25 mg/kg.

5. The method of claim 2, wherein week 3 comprises session 5 and session 6, and wherein the dose of ketamine for session 5 is 1.5 mg/kg and the dose of ketamine for session 6 is 1.75 mg/kg.

6. The method of claim 2, wherein week 4 comprises session 7 and session 8, and wherein the dose of ketamine for session 7 is 2 mg/kg and the dose of ketamine for session 8 is 2 mg/kg.

7. The method of claim 2, wherein the dose of ketamine for week 5 is less than, greater than, or equal to the dose of ketamine in at least one of week 1, week 2, week 3, or week 4.

8. The method of claim 2, wherein week 6 comprises session 9, and wherein the dose of ketamine for session 9 is 2 mg/kg.

9. The method of claim 2, wherein the dose of ketamine for week 7 is less than, greater than, or equal to the dose of ketamine in at least one of week 1, week 2, week 3, week 4, week 5, or week 6.

10. The method of claim 2, wherein week 8 comprises session 10, and wherein the dose of ketamine for session 10 is 2 mg/kg.

11. The method of claim 2, wherein week 9 comprises an assessment to determine a maintenance dose of ketamine.

12. The method of claim 1, wherein the one or more weeks correspond to:
a pre-induction phase comprising one or more appointments for a medical evaluation of the subject and therapist preparation;
an induction phase comprising week 1, week 2, and week 3;
a step-down phase comprising week 4, week 5, and week 7; and
a maintenance phase comprising at least week 10.

13. The method of claim 12, wherein week 1 comprises session 1 and session 2, and wherein the dose of ketamine for session 1 is 0.5 mg/kg and the dose of ketamine for session 2 is 0.75 mg/kg.

14. The method of claim 12, wherein week 2 comprises session 3 and session 4, and wherein the dose of ketamine for session 3 is 0.75 mg/kg and the dose of ketamine for session 4 is 1.0 mg/kg.

15. The method of claim 12, wherein week 3 comprises session 5 and session 6, and wherein the dose of ketamine for session 5 is 1.0 mg/kg and the dose of ketamine for session 6 is at least 1.25 mg/kg.

16. The method of claim 12, wherein week 4 comprises session 7, and wherein the dose of ketamine for session 7 is at least 1.25 mg/kg.

17. The method of claim 12, wherein week 5 comprises session 8, and wherein the dose of ketamine for session 8 is at least 1.25 mg/kg.

28. The method of claim 12, wherein week 7 comprises session 9, and wherein the dose of ketamine for session 9 is at least 1.25 mg/kg.

19. The method of claim 12, wherein week 10 comprises session 10, and wherein the dose of ketamine for session 10 is at least 1.25 mg/kg.

20. The method of claim 1, wherein the at least one baseline assessment is selected from the group consisting of a baseline rating scales for monitoring clinical progression and response to treatment of depression, standard depression rating scales, the Hamilton Depression Rating Scale (HAM-D), the Montgomery—Åsberg Depression Rating Scale (MADRS), the Patient Health Questionnaire 9 (PHQ-9), suicide scales, the Columbia Suicide Severity Rating Scale (C-SSRS), the Scale for Suicidal Ideation (SSI), standard anxiety scales, the General Anxiety Disorder 7 (GAD-7), the Beck Anxiety Inventory (BAI), the Yale-Brown Obsessive Compulsive Scale (YBOCS) for obsessive-compulsive disorder (OCD), the Holmes-Raha Life Stess Inventory, cognitive performance evaluations, depression evaluations, anxiety evaluations, suicide scale evaluations, the post-traumatic stress disorder (PTSD) checklist civilian version (PCL-C), the Clinician-Administered PTSD Scale for Diagnostic and Statistical Manual of Mental Disorders 5th Edition (CAPS-5), the quick inventory of depressive symptomatology (QIDS), a complete blood count panel, a metabolic panel, thyroid stimulating hormone (TSH) with reflex to T4 testing, B12 testing, folate testing, homocysteine testing, vitamin D testing, methylenetetrahydrofolate reductase (MTHFR) gene level testing, enzyme-linked immunoassay (ELISA) testing for brain-derived neurotrophic factor (BDNF) val/val homozygotes, val66met or rs6265, single nucleotide polymorphisms (SNP) testing, c-reactive protein (CRP) testing, interleukin 6 (IL-6) testing, tumor necrosis factor alpha (TNF-a) testing, plasma adiponectin level testing, serum kynurenic acid level testing, kynurenic to quinolinic acid ratio testing, D-serine level testing, a ketamine dissociation scale, and combinations thereof.

21. The method of claim 1, further comprising:
collecting treatment-related data from the subject before, during, or after at least one of the one or more sessions; and
wherein the treatment-related data comprises at least one of information obtained during the at least one baseline assessment or progress of the subject based, at least in part, on an assessment of the subject before, during, or after each session of the one or more sessions.

22. The method of claim 21, further comprising identifying potential candidates for the ketamine-assisted psychotherapy plan based, at least in part, on the treatment-related data.
